(19) United States
(12) Patent Application Publication
(10) Pub. No.: US 2025/0372210 A1
(43) Pub. Date: Dec. 4, 2025

(54) SYSTEM, METHOD, AND PROGRAM FOR RECOGNIZING A POLYMER MOLECULAR STRUCTURE FORMULA USING ARTIFICIAL INTELLIGENCE

(71) Applicant: LG MANAGEMENT DEVELOPMENT INSTITUTE CO., LTD., Seoul (KR)

(72) Inventors: Seungjun LEE, Gimpo-si (KR); Ahra JO, Seoul (KR); Soonyoung LEE, Seoul (KR)

(21) Appl. No.: 19/299,300

(22) Filed: Aug. 13, 2025

Related U.S. Application Data
(63) Continuation of application No. PCT/KR2025/001495, filed on Jan. 24, 2025.

(30) Foreign Application Priority Data
Jan. 24, 2024 (KR) 10-2024-0010891

(51) Int. Cl. G16C 20/20; G16C 20/70; G16C 20/80

(52) U.S. Cl. CPC G16C 20/20; G16C 20/70; G16C 20/80

(57) ABSTRACT

A system, method, and program for recognizing a polymer molecular structure formula using artificial intelligence are disclosed. The system includes at least one processor, and at least one memory storing a command or information that causes the at least one processor to perform an operation, wherein the operation includes detecting a polymer molecular structure formula image to generate detection data, the detection data including information about atomic regions including atoms, bonding between the atoms, and a bracket pair with an associated subscript, the system further including inputting the detection data to each of a first model and a second model to output first cluster data from the first model and to output from the second model second cluster data including group information about the bracket pair and the associated subscript and including information different from the first cluster data.

### CROSS REFERENCE TO RELATED APPLICATIONS

This application is a Bypass Continuation of International Patent Application No. PCT/KR2025/001495, filed on Jan. 24, 2025, which claims priority from and the benefit of Korean Patent Application No. 10-2024-0010891, filed on Jan. 24, 2024, each of which is hereby incorporated by reference for all purposes as if fully set forth herein.

### BACKGROUND

### Field of the Invention

Embodiments of the invention relate generally to a system, a method, and a program for recognizing a polymeric molecular structure, and, more particularly, to a system, a method, and a program that can produce a machine-readable polymer representation from a two-dimensional polymer molecular structure formula image using artificial intelligence (AI).

### Discussion of the Background

A structural formula refers to a graphical representation of a chemical structure or a molecular structure. The structural formula may show in a convenient commonly accepted notation how atoms are arranged in a three-dimensional space occupied by the chemical structure or molecular structure. The structural formula can also indicate clearly or implicitly the chemical bonding of the atoms within a molecular structure.

A polymer refers to a substance having a relatively high molecular weight, the substance being formed by the sequential linking of a plurality of monomer molecules, where monomers are compounds having a relatively low molecular weight. When the polymer is represented by a molecular structure formula, the polymer can be denoted by attaching an arbitrary number of repetitions (n, m, etc., a subscript in the form of a variable representing a degree of polymerization) to a repeated monomer.

The molecular structure formula can be conveniently provided in the form of a two-dimensional image in various documents, papers, etc. Since the molecular structural formulas of polymers are provided throughout much of the chemical literature in the form of two-dimensional images that are not necessarily rendered in a single consistent format, it is difficult to identify or select a particular molecular structural formula consistently and/or exhaustively through general search using, for example, commonly available computer search engines. In particular, given that the polymer of interest can be a copolymer including two or more different monomers, there is a need in the art for improved machine recognition of a polymer molecular structure formula image and association thereof with a chemical structure or molecular structure.

The above information disclosed in this Background section is only for understanding of the background of the inventive concepts, and, therefore, it may contain information that does not constitute prior art.

### SUMMARY OF THE INVENTION

Improved machine recognition of polymer molecular structure formulae will have many useful applications in science and industry. Improved polymer property prediction based on a proposed polymer structure will be possible. AI methods incorporating this advance can be used to develop better polymer synthetic methods. New polymers that fill specific niche applications by having especially suitable sets of properties can be made accessible for the first time. Machine-readable polymer representations that take into account the ways in which those polymers were synthesized and processed will facilitate great improvement in many polymer technologies.

An object of the present invention is directed to providing a system, a method, and a program that can accurately discern a polymer molecular structure by selecting and recognizing necessary information from a two-dimensional polymer molecular structure formula image.

Additional features of the inventive concepts will be set forth in the description which follows, and in part will be apparent from the description, or may be learned by practice of the inventive concepts.

In one aspect, the present invention provides a system for recognizing a polymer molecular structure formula using artificial intelligence that can include at least one processor, and at least one memory storing a command or information that causes the at least one processor to perform an operation, wherein the operation performed by the command or the information can include detecting a polymer molecular structure formula image to generate detection data including information about a bracket pair and a subscript associated with the bracket pair, and inputting the detection data to each of a first model and a second model to output first cluster data from the first model and to output from the second model second cluster data including group information about the bracket pair and the subscript associated with the bracket pair, where the second cluster data includes information different from information provided by the first cluster data.

In some embodiments, the polymer can include two or more different monomers.

In some embodiments, the first cluster data can include at least one of class information and coordinate information of the bracket pair and the associated subscript.

In some embodiments, the second model can include a metric function.

In various embodiments, the detecting of the polymer molecular structure formula image to generate the detection data including the information about the bracket pair and the associated subscript can include inputting the polymer molecular structural formula image into a detector to output the detection data, and the detector can include a detection transformer (DETR) or a deformable DETR.

In some embodiments, the detection data can include an embedding vector.

In some embodiments, at least one of the first model and the second model can include a matrix including the embedding vector.

In some embodiments, the group information about the bracket pair and the associated subscript can include information about which bracket pair and associated subscript among the detected bracket pairs and associated subscripts are included in a group corresponding to each monomer.

In various embodiments, the operation performed by the command or the information can further include converting the structural formula image into a predetermined string format including simplified molecular input line entry system (SMILES) based on cluster data and outputting the converted structural formula image.

In some embodiments, the operation performed by the command or the information can further include acquiring information about a plurality of atomic regions from the polymer molecular structure formula image and acquiring information about bonding relationships between a plurality of atoms based on the information about the plurality of atomic regions.

In another aspect, the present invention provides a method for recognizing a polymer molecular structure formula using artificial intelligence, performed by at least one processor, the method comprising detecting a polymer molecular structure formula image to generate detection data including information about a bracket pair and an associated subscript, and inputting the detection data to each of a first model and a second model to output first cluster data from the first model and to output from the second model second cluster data including group information about the bracket pair and the associated subscript, where the second cluster data includes information different from information provided by the first cluster data.

In some embodiments, the polymer can include two or more different monomers.

In some embodiments, the group information about the bracket pair and the associated subscript can include information about which bracket pair and associated subscript among the detected bracket pairs and associated subscripts are included in a group corresponding to each monomer.

In some embodiments, the outputting of the second cluster data from the second model can include generating a matrix that projects the detection data into another feature space.

A program stored in a computer-readable recording medium according to certain embodiments of the present invention can be stored in a computer-readable recording medium to execute the inventive method for recognizing the polymer molecular structure formula according to embodiments of the present invention.

According to embodiments of the present invention, by simultaneously including a first model that outputs class information and coordinate information from detection data of bracket pairs and associated subscripts of a polymer molecular structure formula and a second model that outputs group information about the bracket pairs and the associated subscripts, it is possible to more accurately recognize a copolymer molecular structure formula that includes two or more different monomers.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a schematic diagram of a system that can implement a method for recognizing a polymer molecular structure formula according to one embodiment of the present invention.

FIG. 2 is a block diagram for describing a configuration of a device that performs the method for recognizing a polymer molecular structure formula according to one embodiment of the present invention.

FIG. 3 is a flowchart for describing a method for recognizing a polymer molecular structure formula according to embodiments of the present invention.

FIG. 4 is a block diagram for describing the method for recognizing a polymer molecular structure formula according to embodiments of the present invention.

FIG. 5 shows molecular structure formulas for illustratively describing a method for recognizing a polymer molecular structure formula according to embodiments of the present invention.

FIG. 6 is a schematic diagram illustratively describing metric learning that can be used in a method for recognizing a polymer molecular structure formula according to embodiments of the present invention.

FIG. 7 is a diagram for describing a structural formula image according to embodiments of the present invention.

FIG. 8 is a diagram for describing an atomic region recognition model according to embodiments of the present invention.

FIG. 9 is a diagram for describing information about a plurality of atomic regions according to embodiments of the present invention.

FIG. 10 is a diagram for describing a method for acquiring information about bonding relationships according to embodiments of the present invention.

FIG. 11 is a diagram for describing a bonding relationship recognition model according to embodiments of the present invention.

### DETAILED DESCRIPTION

The present inventors have unexpectedly found unique ways to use artificial intelligence to encode polymer molecular structure formulas in machine-readable formats that provide entrée into the vast possibilities available in the blossoming field of polymer cheminformatics. Using these machine-readable formats, the awesome capabilities of contemporary computers can now be brought to bear to advance many important inquiries in polymer science.

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As is customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

A system for recognizing a molecular structure formula according to the present invention may include a device, and the device may include all kinds of devices that can perform computational processing and provide results to a user. For example, a system for recognizing a molecular structure formula according to the present invention may include at least one of a computer, a server device, and a portable terminal, or may be implemented in any one form having the same or similar functions thereof.

In addition, the system for recognizing the molecular structure formula according to the present invention may include being implemented in the form of a service provided to a user terminal in a form of a web service or the like from a server, or in a form in which the server and the user terminal are linked.

However, the method for implementing the system for predicting the properties of a material according to the present invention is not limited thereto, and other forms of user access to systems that can be implemented as described herein may be included in the present invention.

*Here, the computer may include, for example, a notebook, a desktop, a laptop, a tablet PC, a slate PC, etc., any of which can be equipped with a web browser.

The server device can be a server that processes information in communication with an external device, and may include an application server, a computing server, a database server, a file server, a game server, a mail server, a proxy server, and a web server. The portable terminal can be, for example, a wireless communication device ensuring portability and mobility and may include all kinds of handheld-based wireless communication devices such as a personal communication system (PCS), a global system for mobile communications (GSM), a personal digital cellular (PDC), a personal handyphone system (PHS), a personal digital assistant (PDA), an international mobile telecommunication-2000 (IMT-2000), a code division multiple access-2000 (CDMA-2000), a w-code division multiple access (W-CDMA), a wireless broadband internet (WiBro) terminal, a smart phone, and wearable devices such as a watch, a ring, a bracelet, an anklet, a necklace, glasses, contact lenses, or a head-mounted device (HMD).

AI models according to embodiments of the present invention may be controlled, executed, learned, driven, etc., by at least one processor, and therefore, at least one of the tasks of executing, learning, and driving the AI models may be performed by at least one processor. The AI models may be stored in a memory.

In addition, according to embodiments of the present invention, a command that causes at least one processor to perform an operation may be included in at least one memory.

The at least one processor may cause the AI model to operate, and may also cause other components (e.g., a conversion unit, a calculation unit, etc.) that implement the system to operate in addition to the AI model.

In addition, in the embodiments, the AI model may include an artificial neural network (ANN), a machine learning model, etc.

The ANN is a model used in machine learning, and may refer to a model having problem-solving capability, which model is composed of artificial neurons (nodes) forming a network by synaptic connections. The ANN may be defined by connection patterns between neurons of different layers, a learning process that updates model parameters, and an activation function that generates an output value.

The ANN may include an input layer, an output layer, and optionally, one or more hidden layers. Each layer may include one or more neurons, and the ANN may include the synapses that connect the neurons to other neurons. In the ANN, each neuron may output a function value of an activation function that depends on input signals input through the synapses, weights of the synaptic connections, and bias of the neuron.

Parameters may include the model parameters and hyperparameters, where the model parameters refer to parameters that are changed and determined through learning and may include the weights of the synaptic connections, the biases of the neurons, etc.

Hyperparameters refer to parameters that need to be set before learning in a machine learning algorithm and include a learning rate, a number of iterations, a mini-batch size, an initialization function, etc.

The learning of the ANN may include determining the model parameters that minimize a loss function, where the loss function may be used as an indicator for determining optimal model parameters in the learning process of the ANN.

The machine learning may include supervised learning, unsupervised learning, reinforcement learning, or the like according to a learning method but is not limited thereto. Among the ANNs, the machine learning implemented with a deep neural network (DNN) including a plurality of hidden layers is called deep learning, and the deep learning is included as a part of the machine learning.

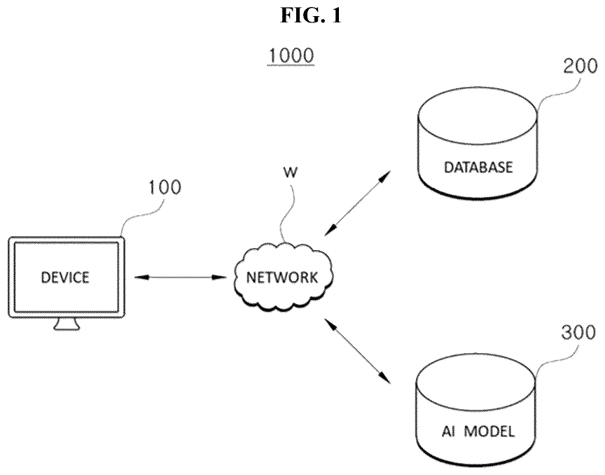

FIG. 1 is a schematic diagram of a system that can implement a method for recognizing a polymer molecular structure formula according to one embodiment of the present invention.

As shown in FIG. 1, a system 1000 may include a device 100, a database 200, and an AI model 300.

The device 100, the database 200, and the AI model 300 that are included in the system 1000 may perform communication via a network W. Here, the network W may include a wired network and a wireless network. For example, the network may include various networks such as a local area network (LAN), a metropolitan area network (MAN), and a wide area network (WAN).

In addition, the network W may also include the well-known world wide web (WWW). However, the network W according to the present invention is not limited to the above-listed networks and may include, at least in part, a well-known wireless data network, a well-known telephone network, or a well-known wired and wireless television network.

The device 100 may acquire or output feature data on information about a polymer molecular structure formula based on the AI model 300. The feature data may include a feature vector, embedding, or the like.

The database 200 may store various training data for training the AI model 300. In addition, the database 200 may store a polymer molecular structure formula image, a ground-truth (GT) value, a training set for training, etc., and, in various embodiments, may store output data output by the AI model 300. However, the system 1000 may not include the database 200 when the training of the AI model 300 is completed.

FIG. 1 shows a case in which the database 200 is implemented outside the device 100. In this case, the database 200 may be connected to the device 100 in a wired or wireless communication manner. However, this is only one embodiment, and the database 200 may also be implemented as one component of the device 100.

FIG. 1 shows a case in which the AI model 300 is implemented outside the device 100 (e.g., implemented in a cloud-based manner), but the present invention is not limited thereto, and the AI model 300 may be implemented as one component of the device 100.

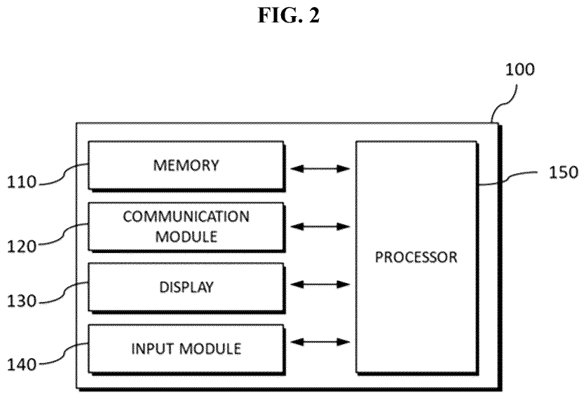

FIG. 2 is a block diagram for describing a configuration of a device that performs the method for recognizing the polymer molecular structure formula according to one embodiment of the present invention.

As shown in FIG. 2, the device 100 may include a memory 110, a communication module 120, a display 130, an input module 140, and a processor 150. However, the present invention is not limited thereto, and software and hardware components of the device 100 may be modified/added/omitted according to a required operation within a scope obvious to those skilled in the art. In addition, the device 100 may be replaced with a system, and the device 100 may include a plurality of devices and in this case, each component included in the device 100 may be included in at least one of the plurality of devices.

The memory 110 may store data supporting various functions of the device 100 and a program for the operation of the processor 150, may store input/output data, may store a plurality of application programs or applications that are driven on the present device, and may store data, commands and the AI model for the operation of the device 100. At least some of the application programs may be downloaded from an external server via wireless communication. The memory 110 may store a command or information that causes the processor 150 to perform an operation.

Such memory 110 may include at least one type of storage medium selected from a flash memory type, a hard disk type, a solid state disk type (SSD type), a silicon disk drive type (SDD type), a multimedia card micro type, a card-type memory (e.g., an SD or XD memory), a random access memory (RAM), a static random access memory (SRAM), a read-only memory (ROM), an electrically erasable programmable ROM (EEPROM), a programmable ROM (PROM), a magnetic memory, a magnetic disk, and an optical disk.

In addition, the memory 110 may include a database that may be separate from the present device and connected in a wired or wireless communication manner. The database 200 shown in FIG. 1 may be implemented as one component of the memory 110.

The communication module 120 may include one or more components that enable communication with an external device, and may include at least one of, for example, a broadcasting reception module, a wired communication module, a wireless communication module, a short-range communication module, or a position information module.

The wired communication module may include not only various wired communication modules such as a local area network (LAN) module, a wide area network (WAN) module, and a value added network (VAN) module, but also various cable communication modules such as a universal serial bus (USB), a high definition multimedia interface (HDMI), a digital visual interface (DVI), a recommended standard 232 (RS-232), power line communication, and a plain old telephone service (POTS).

In addition to the WiFi module and the wireless broadband (WiBro) module, the wireless communication module may include a wireless communication module for supporting various wireless communication methods such as global system for mobile communication (GSM), code division multiple access (CDMA), wideband CDMA (WCDMA), universal mobile telecommunications system (UMTS), time division multiple access (TDMA), long term evolution (LTE), fourth generation wireless technology (4G), fifth generation wireless technology (5G), or sixth generation wireless technology (6G).

The display 130 can display (outputs) information or data that are processed in the device 100, data that are input or output through the AI model 300, etc. In addition, the display 130 may display execution screen information of an application program (e.g., an application) driven on the device 100, or user interface (UI) or graphic user interface (GUI) information according to such execution screen information.

The input module 140 can be for receiving information from a user, and when receiving information through a user input unit, the processor 150 may control the operation of the device 100 to correspond to the input information.

Such input module 140 may include a hardware physical key (e.g., a button located on at least one of a front surface, a back surface, and a side surface of the present device, a dome switch, a jog wheel, a jog switch, etc.) and a software touch key. As an example, the touch key may be formed as a virtual key, a soft key, or a visual key that is displayed on a touchscreen type of the display 130 through software processing or may be formed as the touch key disposed on a portion of the input module other than the touchscreen. The virtual key or visual key can have various forms, can be displayed on the touchscreen and can be formed as, for example, a graphic, text, an icon, a video, or a combination thereof.

The processor 150 may be implemented with a memory that stores data for an algorithm for controlling the operations (including learning or execution of the AI model) of the components in the device 100, or the processor 150 may be implemented by a program that reproduces this algorithm and uses it within at least one other processor (not shown) that performs the operations of the components in the device 100 using the data stored in the memory. The memory and the processor may each be implemented as separate chips or may be implemented as a single chip.

The processor 150 may be one or more processors and/or processing circuits for executing program codes and controlling basic operations of the device 100. The processor may include a central processing unit (CPU), a graphic processing unit (GPU), a field-programmable gate array (FPGA), an application-specific integrated circuit (ASIC), a complex programmable logic device (CPLD), a dedicated circuit for implementing functions, a special-purpose processor for implementing neural network-based processing, or a system including other systems.

In some embodiments, the system 1000 or the device 100 according to the present invention may include at least one processor, and, when including a plurality of processors, the plurality of processors may be included in different devices 100.

The processor 150 may control the operations of the components in the device 100 by combining any one or a plurality of the above-described components in order to implement various embodiments according to the present invention, the embodiments being described below, on the device 100.

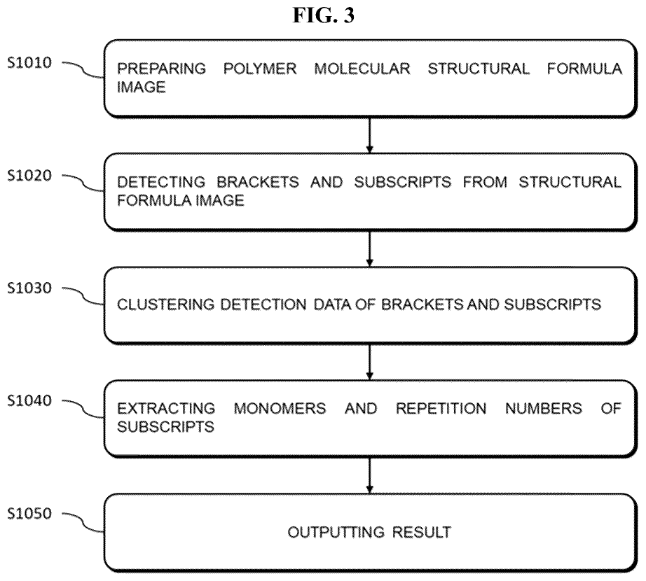

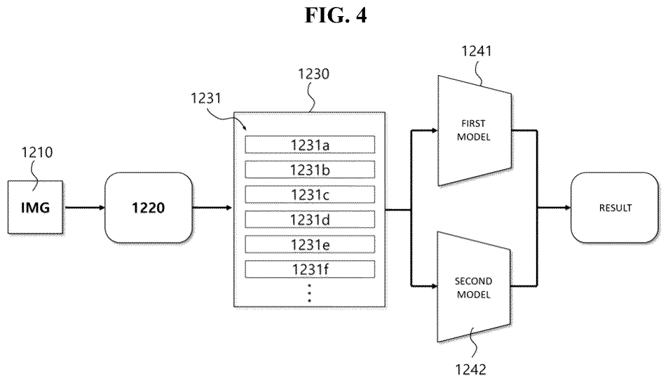

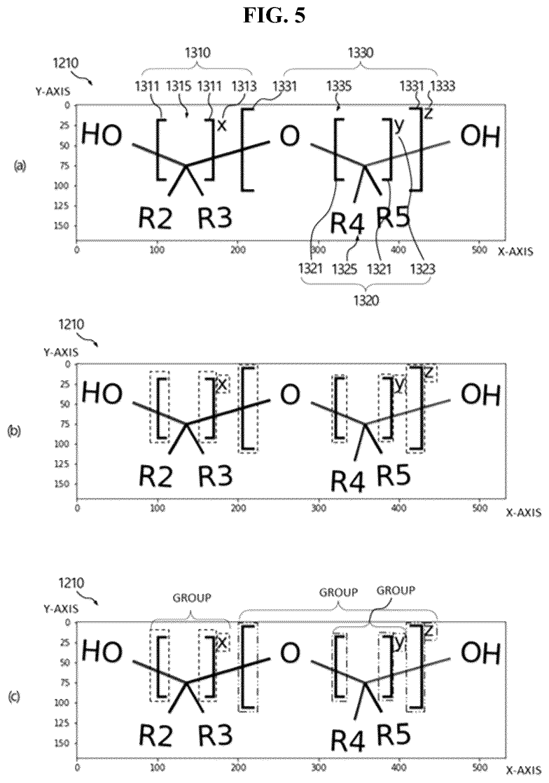

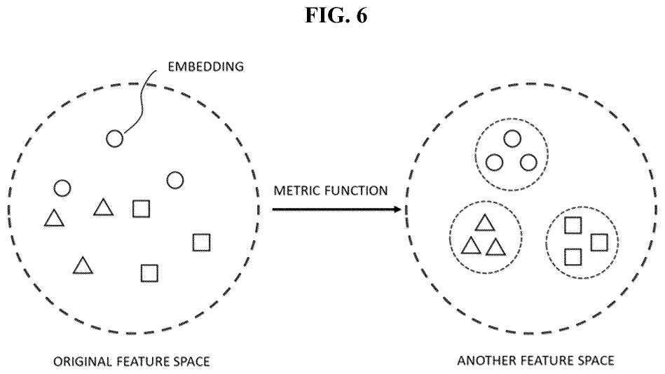

FIG. 3 is a flowchart for describing a method for recognizing a polymer molecular structure formula according to embodiments of the present invention, FIG. 4 is a block diagram for describing a method for recognizing a polymer molecular structure formula according to embodiments of the present invention, and FIG. 5 shows molecular structure formulas for illustratively describing a method for recognizing a polymer molecular structure formula according to embodiments of the present invention. FIG. 6 is a schematic diagram illustratively describing metric learning that may be used in a method for recognizing a polymer molecular structure formula according to embodiments of the present invention.

Referring to FIGS. 3 and 4, a method for recognizing the polymer molecular structural formula according to embodiments includes preparing a polymer molecular structure formula image (S1010), detecting bracket pairs and associated subscripts from a polymer molecular structure formula image to generate detection data (S1020), clustering the bracket pairs and the associated subscripts in the detection data to generate cluster data (S1030), extracting monomers and repetition numbers of subscripts using the data detected from the polymer molecular structure formula image (S1040) and outputting a result (S1050).

The method for recognizing a polymer molecular structure formula according to embodiments may be performed by at least one processor. Commands or information that cause the at least one processor to perform an operation may be included in at least one memory.

The at least one processor or the at least one memory may be included in a plurality of devices. In addition, the method for recognizing the polymer molecular structure formula according to embodiments may be performed by the operation performed by the commands or information and may include performing iterative learning.

Hereinafter, each operation will be described in detail.

First, the method for recognizing the polymer molecular structure formula according to embodiments may include preparing a polymer molecular structure formula image 1210 (S1010). The polymer molecular structure formula image 1210 may include the polymer molecular structure formula including at least one monomer and may include a copolymer molecular structure formula including two or more different monomers. For example, as shown in FIG. 5A, the polymer molecular structure formula image 1210 may include a copolymer molecular structure formula including three different monomers 1315, 1325, and 1335. The step of preparing a polymer molecular structure formula image 1210 may be

performed by at least one processor, and the commands or the information packets that cause the at least one processor to perform the operation may be stored in at least one memory. The step of preparing the polymer molecular structural formula image 1210 may further include storing the polymer molecular structural formula image 1210 in a memory that can be included in the processor or a separate volatile or non-volatile memory, or the like. The polymer molecular structure formula image 1210 may include an image stored in the memory, an image received from the outside through a network or a communication module and stored, an image received from the outside through the network or the communication module in real time and streamed, etc. However, the present invention is not limited thereto and may include all methods and approaches in which the polymer molecular structure formula image is prepared so that the polymer molecular structural formula image 1210 may be recognized through the operation performed by at least one processor.

The polymer molecular structure formula image 1210 may be an image in which the structural formula is represented in a graphical form. The structural formula may refer to a graphical representation of a chemical structure or a molecular structure. The structural formula may include information about the arrangement of atoms in a three-dimensional space and may include information about chemical bonding between the atoms. The polymer molecular structure formula image 1210 may include an annotation, and for example, the annotation may refer to a name of a compound, etc.

Referring to the example of FIG. 5, as shown in FIG. 5A, the polymer molecular structural formula image 1210 includes first to third monomers 1315, 1325, and 1335, and each monomer is represented by bracket pairs 1311, 1321, and 1331 and associated subscripts 1313, 1323, and 1333 that refer to the number of repetitions of the corresponding monomer. The polymer molecular structure formula image 1210 may include a first group 1310 including the first monomer 1315, the bracket pair 1311 distinguishing the first monomer 1315, and the subscript 1313, and may further include second and third groups 1320 and 1330 that include the second monomer 1325 and the third monomer 1335, respectively.

Here, the bracket pair refers to a notation for defining or distinguishing a unit of a repeated monomer in a polymer. That is, the molecule inside the brackets represents a structure of each monomer, and a line coming out of the brackets represents how the terminal monomers of the monomer chain are bonded to other atoms or molecules. The subscript refers to the number of repetitions of the monomer, and when characters such as n or m are used as the subscript, they represent that the indicated monomer is repeated n or m times.

Next, the bracket pairs and the associated subscripts are detected from the polymer molecular structural formula image 1210 (S1020).

Referring to FIGS. 3 and 4, the step of detecting the bracket pairs and the associated subscripts from the polymer molecular structure formula image 1210 may include detecting the polymer molecular structure formula image 1210 and outputting detection data 1230 about the bracket pairs and the associated subscripts. The detection data 1230 may include information about the bracket pairs and the associated subscripts. In addition, the detection data 1230 about the bracket pairs and the associated subscripts may include at least one embedding 1231, and the embedding may include an embedding vector that is the form of a vector.

The step of detecting the polymer molecular structural formula image 1210 may include inputting the image into a detector 1220 and outputting the detection data 1230 about the bracket pairs and the associated subscripts. The detector 1220 may include an object detector of transformer series and include a detection transformer (DETR) or a deformable DETR, but the present invention is not limited thereto.

Referring to the example of FIG. 5, as shown in FIG. 5B, the brackets (dotted lines) and the subscripts (dash-dotted lines) may each be detected by detecting the polymer molecular structure formula image 1210.

Meanwhile, each of the at least one embedding 1231 included in the detection data 1230 about the bracket pairs and the associated subscripts may be an embedding including each packet of individual information of the polymer molecular structural formula image 1210. The embedding 1231 may include an embedding vector including information about the bracket and the embedding vector including information about the subscript. The embedding vector may be, for example, the embedding vector of a length of 1024.

The embedding 1231 may include individual embedding including information about each of the bracket pairs 1311, 1321, 1331 and the associated subscripts 1313, 1323, 1333 that refer to the number of repetitions of the corresponding monomer within the polymer molecular structure image, and the individual embedding may include the embedding vector.

The embedding 1231 may each include one embedding vector including information about a left bracket and another embedding vector including information about a right bracket, which constitute a bracket pair.

Referring to the example of FIG. 5, the polymer molecule included in the polymer molecular structure formula image 1210 shown in FIG. 5A may include the first to third bracket pairs 1311, 1321, and 1331 and the first to third associated subscripts 1313, 1323, and 1333, and the information about each of the brackets and the subscripts may each correspond to separate embedding vectors.

For example, in embodiments, the embedding 1231 may include an embedding vector 1231a including information about the left first bracket 1311, an embedding vector 1231b including information about the right first bracket 1311, and an embedding vector 1231c including information about the first subscript 1313. In addition, the embedding 1231 may include embedding vectors 1231d and 1231e that include information about each of the left and right brackets 1321 of the second group 1320 and an embedding vector 1231f including information about the second subscript 1323. The step of detecting the bracket pairs and the associated subscripts from the polymer molecular structure formula image 1210 may also include recognizing remaining parts other than the brackets and the subscripts in the polymer molecular structure formula image, the remaining parts including the atoms constituting the polymer molecule included in the polymer molecular structure formula image 1210 and bonding relationships between the atoms. In addition, the step of detecting the atoms constituting the polymer molecule included in the polymer molecular structure formula image 1210 and the bonding relationships between the atoms may include detecting the atoms constituting each monomer and the bonding relationships between those atoms and between those atoms and other atoms.

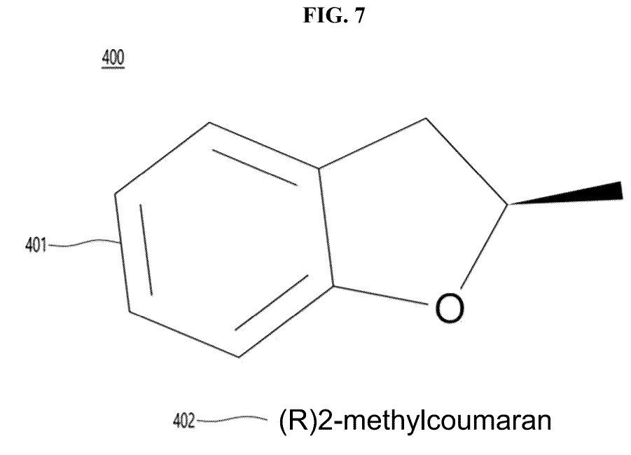

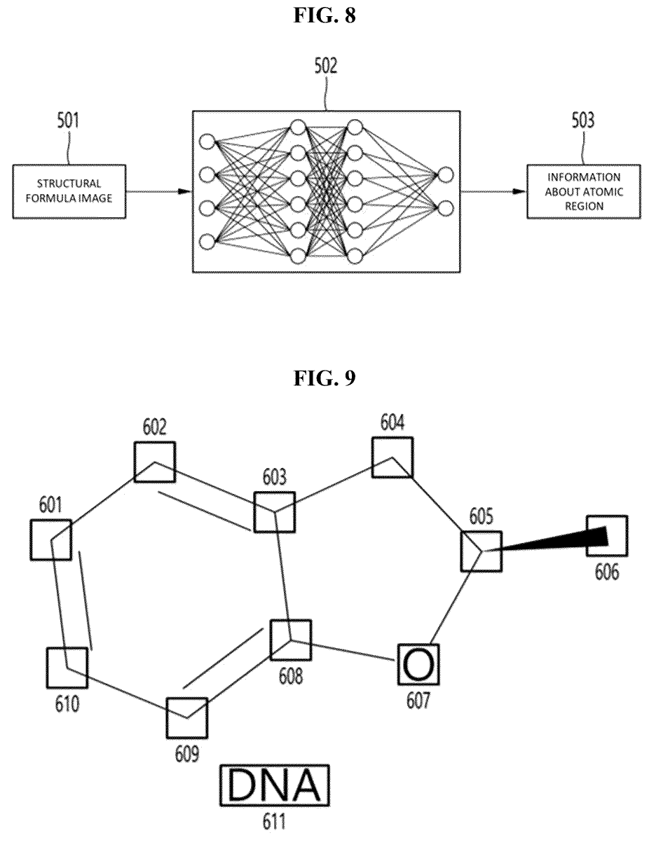

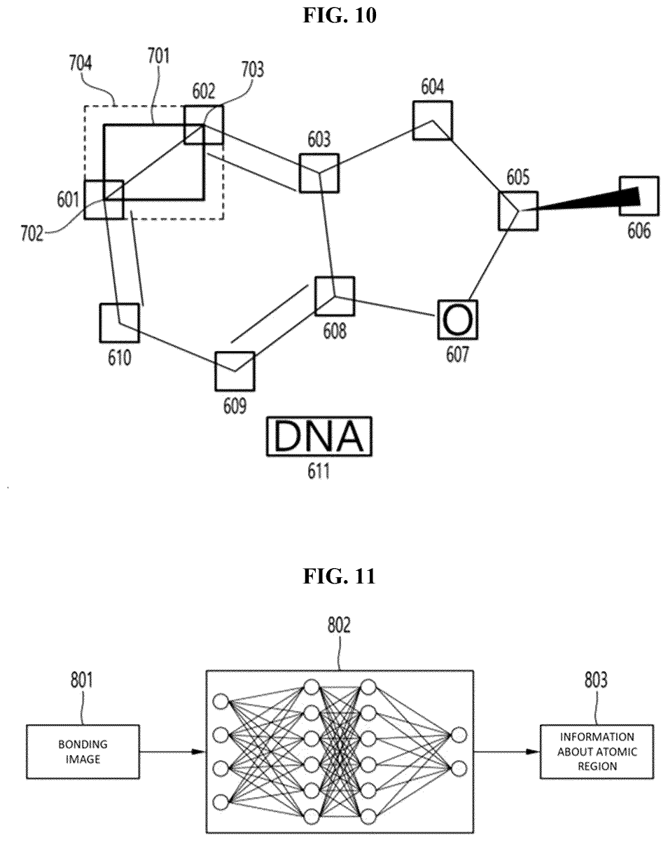

A method for detecting the atoms constituting the polymer molecule included in the polymer molecular structure formula 1210 and the bonding relationships between the atoms according to various embodiments of the present invention will be described in more detail with reference to FIGS. 7 to 11. FIG. 7 is a diagram for describing a structural formula image according to embodiments of the present invention, FIG. 8 is a diagram for describing an atomic region recognition model according to embodiments of the present invention, FIG. 9 is a diagram for describing information about a plurality of atomic regions according to embodiments of the present invention, FIG. 10 is a diagram for describing a method for acquiring information about bonding relationships according to embodiments of the present invention, and FIG. 11 is a diagram for describing a bonding relationship recognition model according to embodiments of the present invention.

First, referring to FIG. 7, a structural formula image 400 may include a structural formula 401 that graphically represents a molecular structure. In addition, the structural formula image 400 may include an annotation 402. When the structural formula image 400 is generated, the annotation may be imaged and included as a description or a name of the molecular structure in the structural formula image. When recognizing the structural formula image 400 and converting the structural formula image 400 into a predetermined string format, it is necessary to filter the structural formula 401 alone as a recognition target, excluding the annotation 402 included in the structural formula image 400.

The information about the atomic region may include at least one of identification information about the atomic region, information about an atomic position, and information about the atom in the structural formula image. The identification information about the atomic region may refer to identification information (or a number) that may distinguish each of a plurality of atomic regions recognized in the structural formula image.

In addition, the information about the atomic position may refer to coordinate information corresponding to the atomic region in the structural formula image. For example, when the atomic region is represented as a quadrangle, the information about atomic position may include a coordinate of each vertex of the quadrangle corresponding to the atomic region and a coordinate of a center point of the quadrangle.

In addition, the information about the atom may include information about an element symbol of the atom corresponding to the atomic region. For example, when an image corresponding to the atomic region is represented as a vertex, carbon (C) may correspond to the information about the atom. In addition, when the element symbol (e.g., oxygen (O)) is described in the image corresponding to the atomic region, information about the corresponding element symbol (O) may correspond to the information about the atom.

Meanwhile, the information about the atomic region may include information about reliability of the information about atomic region acquired from the structural formula image. For example, the processor may acquire the information about reliability of information about the atomic region acquired from the structural formula image as a value between 0.00 and 1.00, and may use only the information about the atomic region having the information about reliability equal to or greater than a predetermined value.

Referring to FIG. 8, the processor may input a structural formula image 501 to an atomic region recognition model 502 and acquire the information about the atomic region output from the atomic region recognition model 502.

The atomic region recognition model 502 may be an artificial neural network (ANN) trained to output information about at least one atomic region 503 included in the image with respect to the input structural formula image 501. The ANN is a model used in machine learning, and may refer to a model having problem-solving capability, which is composed of artificial neurons (nodes) forming a network by synaptic connections. For example, the atomic region recognition model 502 may be an ANN model based on a convolutional neural network (CNN).

The processor may train the atomic region recognition model 502 composed of the ANN using learning data about the structural formula image and the information about the atomic region. Meanwhile, the atomic region recognition model 502 may be a model trained by the processor.

The trained atomic region recognition model 502 may be stored in a memory or stored in a storage unit of a server. The processor may cause the model stored in the memory, etc. to perform an operation.

Referring to FIG. 9, the processor may acquire information about a plurality of atomic regions 601, 602, 603, 604, 605, 606, 607, 608, 609, 610, and 611 that are output from the atomic region recognition model 502. The information about a plurality of atomic regions may include information about vertex regions 601, 602, 603, 604, 605, 606, 608, 609, and 610 in the structural formula, an element symbol region 607 in which the element symbol is described, and an annotation region 611. For example, since the atomic region recognition model 502 may be trained to output the element symbol region 607 in which the element symbol is described, there may also be a case in which the atomic region recognition model 502 outputs the annotation region 611 as the information about the atomic region. Therefore, there may be a need to filter the information about the annotation region from the information about the plurality of atomic regions. For example, since the annotation region 611 is not shown to have a bonding relationship with other atomic regions, when there is no bonding relationship between the annotation region 611 and other atomic regions, the processor 180 may classify the annotation region 611 as an annotation. Meanwhile, the processor may acquire information about bonding relationships between the plurality of atoms based on the information about the plurality of atomic regions.

The processor may acquire the information about the bonding relationship of each atomic region with another atomic region based on the information about the plurality of atomic regions. The processor may acquire a bonding image between a first atom and a second atom based on first atomic region information and second atomic region information among the information about the plurality of atomic regions, and acquire the information about the bonding relationship of the first atom and the second atom based on the acquired bonding image.

Referring to FIG. 10, a method for acquiring the information of the bonding relationship will be described, in which method the processor may select a first atomic region information 301 from among the information about the plurality of atomic regions. In addition, the processor may select a second atomic region information 602 different from the first atomic region information. The processor may then follow up by acquiring a bonding image 701 between the first atom and the second atom based on first atomic position information 702 of the first atomic region information 601 and second atomic position information 703 of the second atomic region information 602. In this case, the first atomic position information 702 and the second atomic position information 703 may be a center point position of each the atomic region. However, the first atomic position information 702 and the second atomic position information 703 are not limited to the center point positions of the atomic regions.

The processor may acquire the bonding image 701 including the center point position of each atomic region based on the first atomic position information 702 of the first atomic region information 601 and the second atomic position information 703 of the second atomic region information 602. In addition, the processor may acquire a bonding image 704 including the first atomic region and the second atomic region based on the first atomic region information 601 and the second atomic region information 602. A size and shape of the bonding image may be variously adjusted.

The processor may acquire the bonding images between combinable atoms for each of the plurality of atomic regions. However, when acquiring the bonding images between all combinable atoms, the amount of computation involved may be considered to be excessive. Therefore, a practitioner may decide that the processor should acquire only the bonding image between a first atom and a second atom where the two atoms are positioned within a predetermined distance from each other based on the information about the plurality of atomic regions.

For example, the processor may select a second atomic region positioned within a predetermined distance from a first atomic region based on the first atomic region information among the information about the plurality of atomic regions. Referring to FIG. 10, the processor may specify the second atomic regions 602, 603, 608, 609, and 610 that are positioned within a predetermined distance from the first atomic region 601 and acquire the bonding image between the first atom 601 and each of the second atoms to acquire the information about the bonding relationships between the first atom and the second atoms.

In contrast, the processor may determine that third atomic regions 604, 605, 606, 607, and 611 that are positioned outside a predetermined distance from the first atomic region 601 have no bonding relationship with the first atomic region. In this way, the amount of computation may be decreased.

Meanwhile, the processor may acquire the information about the bonding relationships between the atoms based on the acquired bonding images. For example, the processor may input the bonding images into a bonding relationship recognition model and acquire the information about the bonding relationships output from the bonding relationship recognition model.

FIG. 11 is a diagram for describing the bonding relationship recognition model, and referring to FIG. 11, the processor may input a bonding image 801 into a bonding relationship recognition model 802 and acquire the information about the bonding relationship output from the bonding relationship recognition model 802.

The information about the bonding relationship is information about bonding between the atoms and may include information about non-bonding, single bonding, double bonding, triple bonding, chiral center bonding, etc. Non-bonding may refer to a case in which there is no bonding between the atoms. The chiral center bonding may be depicted by a bond coming toward a viewer from a two dimensional plane in which a polymer molecular structure formula is represented, indicated by a wedge line, or by a bond extending away from the viewer relative to that two dimensional plane, indicated by a dash line. The bonding relationship recognition model 802 may be an artificial neural network (ANN) trained to output information about the bonding relationships 803 with respect to the input bonding image 801. The ANN is a model used in machine learning and may refer to a model having problem-solving capability, which model is composed of artificial neurons (nodes) forming a network by synaptic connections. For example, the bonding relationship recognition model 802 may be an ANN model based on a convolutional neural network (CNN).

The processor may train the bonding relationship recognition model 802 composed of the ANN using learning data about the bonding images and the information about the bonding relationships.

The trained bonding relationship recognition model 802 may be stored in a memory. The processor may use the bonding relationship recognition model 802 stored in the memory.

The processor may then generate an adjacency matrix based on the information about the plurality of atomic regions and the information about the bonding relationships between the plurality of atoms. The processor may generate the adjacency matrix having each of the plurality of atoms as a vertex and having the information about the bonding relationships of each of the plurality of atoms as edges of the matrix.

FIG. 11 is a diagram for describing the adjacency matrix, and, referring to FIG. 11, the processor may generate each of the atoms of the plurality of atomic regions 601 to 611 as a vertex of the adjacency matrix. The processor may complete the adjacency matrix using the information about the bonding relationships of each of the plurality of atoms to form edges. For example, the processor may generate an edge value of the adjacency matrix by mapping each unit of information about the bonding relationship to an arbitrary number representing bond order. For example, the processor may map non-bonding to ‘0’, single bonding to ‘l’, double bonding to ‘2’, and triple bonding to ‘3’. In addition, the processor may map a case of bonding about a chiral center in which a bond extends in a directional manner toward a viewer from a two dimensional plane in which a molecular structure formula is represented, the bond connecting a starting vertex with an arrival vertex, to ‘5’, and the processor may map a case of a bond in the same position but having opposite directionality to ‘0’. If the atomic regions of a molecular structure formula represented in two dimensions as an approximately planar structure are imagined to have horizontal rows and vertical columns intersecting them, the directionality of bonds departing from the plane of the structure is taken to proceed from top to bottom (lower to higher row numbers) and/or from left to right (lower to higher column numbers).

For example, referring to FIG. 10 and FIG. 11, atomic regions 601, 602, 603, 604 and 605 may be understood to reside in each of the first five rows of the structure, with atomic region 606 residing in row 5. Column 1 includes atomic regions 601 and 610, column 2 includes atomic regions 602 and 609, column 3 includes atomic regions 603 and 608, column 4 includes atomic regions 604 and 607, column 5 includes atomic region 605 and column 6 includes atomic region 606. Since a value at the sixth column of the fifth row of the adjacency matrix is at an arrival vertex relative to a starting vertex at the fifth column of the fifth row and the bonding is directional from a first atom 605 to a second atom 606 (in a direction from lower to higher column number), a corresponding value of ‘5’ may be generated. A value at the fifth column of the sixth row of the adjacency matrix, which is the opposite direction, may be generated as ‘0’. Similarly, the processor 180 may map a case in which the bonding is in a direction away from a viewer relative to a plane of a molecular structure formula in the order of increasing row and column numbers to ‘6’, and map a case in the opposite direction to ‘0’.

Usefully, the processor may generate a predetermined string format corresponding to the structural formula image based on the generated adjacency matrix. The processor may acquire the information about the bonding relationships with other atoms by traversing each of the atomic regions corresponding to the vertices of the adjacency matrix. The processor may specify the information about the atom of each of the plurality of atomic regions using the acquired information about the bonding relationships between the atoms and generate a predetermined string format corresponding to the structural formula image based on the information about the plurality of atoms and the information about the bonding relationships between the plurality of atoms.

In this case, the string format may include a mol file format, an sdf file format, etc. that are file formats that may represent information about compounds (e.g., element positions, bonding relationships, etc.). The string format may include information about a simplified molecular input line entry system (SMILES).

The method for detecting the atoms of the polymer molecule, the bonding relationships between the atoms, etc. from the polymer molecular structural formula image according to embodiments of the present invention is not limited to the descriptions with reference to FIGS. 7 to 11, and various methods for detecting the structural formula image may all be used.

Furthermore, in the method for recognizing the polymer molecular structural formula according to embodiments of the present invention, the step of detecting the atoms constituting the polymer molecule and the step of detecting bonding relationships between the atoms that are included in the polymer molecular structural formula image 1210 are not necessarily included in the operation of detecting the bracket pairs and the associated subscripts from the polymer molecular structural formula image 1210, and the step of detecting the atoms constituting the polymer molecule and the step of detecting bonding relationships between the atoms may be included in another operation or implemented as separate operations.

Referring back to FIGS. 3 and 4, subsequently, cluster data is generated by clustering the brackets and the subscripts in the detection data (S1030).

The step of generating the cluster data by clustering the brackets and the subscripts in the detection data may include inputting the detection data into a first model 1241 and a second model 1242 to output first cluster data including at least one of class information and coordinate information of the brackets and the subscripts from the first model 1241 and to output second cluster data including group information about the brackets and the subscripts from the second model 1242. The cluster data may include the first cluster data and the second cluster data.

The first model 1241 and the second model 1242 may be pre-set algorithms or pre-trained models. At least one of the first model 1241 and the second model 1242 may be executed or trained by the processor.

The first cluster data output from the first model 1241 may include information different from the second cluster data output from the second model 1242.

In embodiments, the detection data including the embedding 1231 may be input into the first model 1241 so that the class information and the coordinate information of an object (a bracket, a subscript) corresponding to each embedding 1231 may be output. For example, referring to FIG. 5A, the polymer molecular structural formula image 1210 may be projected on a space having an x-axis and a y-axis, and each detection object (a bracket, a subscript, an atom, an atomic bonding, etc.) of the polymer molecule may have a coordinate according to the x-axis and y-axis of the space.

The first model 1241 may include a linear layer composed of a matrix. In one embodiment, the first model 1241 may include the linear layer including the matrix (e.g., 1024×4) that derives class probabilities for four classes of the bracket, the subscript, the atom, and the carbon (C) from the embedding 1231 and the linear layer including the matrix (e.g., 1024×4) that derives the coordinate information of the detection object such as the bracket and the subscript from the embedding 1231.

The class information output from the first model 1241 may include classification information of the object corresponding to each embedding 1231, that is, information about what type of object the object refers to. For example, when the embedding 1231 about the brackets 1311, 1321, and 1331 is input into the first model 1241 and related information is output, the class information indicating that information about the corresponding embedding 1231 corresponds to ‘bracket’ may be output.

By outputting the first cluster data including the class information and the coordinate information of the brackets and the subscripts through the first model 1241, information about which monomer the brackets and the subscripts correspond to, where the brackets and the subscripts are positioned in the bonding relationships between other atoms, etc. may be output.

In embodiments, the detection data including the embedding 1231 may be input to the second model 1242 so that group information of objects (a bracket, a subscript) corresponding to each embedding 1231 may be output. The group information may include information about which brackets and subscript among the brackets and the subscripts form the respective monomer groups.

The second model 1242 may include a layer that rearranges the input embedding 1231 into another new space. The second model 1242 may include the matrix (e.g., 1024×1024) that projects the input embedding 1231 into the embedding of a different length (e.g., 1024 length). The second model 1242 may include a metric function.

Referring to FIG. 6, the second model 1242 including the metric function may enable the embeddings of an original feature space to be clustered (grouped) in a new feature space. The second model 1242 may include generating the matrix that projects the detection data into a new different feature space.

The second model 1242 may be a model trained by various methods and may be a model trained based on, for example, an infoNCE loss, but is not limited thereto.

For example, the second model 1242 may be executed or trained in a manner that assigns a loss by defining a positive pair and a negative pair using the infoNCE loss. The positive pair corresponds to ground truth, and for example, the first brackets 1311 and the first subscript 1313 included in the first group 1310 corresponding to the first monomer 1315 may output the result as the positive pair. On the other hand, when clustering into one group such as the first brackets 1311 and the second subscript 1323 is not the ground truth, the corresponding negative result is output.

By outputting the second cluster data including the group information about the bracket and the subscript through the second model 1242, information about which brackets and subscripts among the detected brackets and subscripts are included in a group corresponding to which monomer may be output. For example, referring to FIG. 5C, the second model 1242 may output the second cluster data including information that the first brackets 1311 and the first subscript 1313 are one group included in the first group 1310 corresponding to the first monomer 1315, and may output the second cluster data including similar group information for the brackets and the subscripts included in the remaining second and third groups 1320 and 1330.

As described above, according to embodiments of the present invention, the system using the method for recognizing the polymer molecular structure formula according to the present invention may accurately recognize the copolymer molecular structure formula including two or more different types of monomers by simultaneously including the first model 1241 that outputs the class information and the coordinate information from the detection data of the brackets and the subscripts of the polymer molecular structure formula and the second model 1242 that outputs the group information about the brackets and the subscripts.

Referring back to FIGS. 3 and 4, the monomers and repetition numbers of the subscripts that are detected from the polymer molecular structure formula image are extracted (S1040), and a result is output (S1050).

The step of extracting the monomers and the repetition numbers of the subscripts that are detected from the polymer molecular structure formula image may extract the monomers and the repetition numbers of the subscripts from information detected through the method of detecting the atoms constituting the polymer molecule and the bonding relationships between the atoms, but the present invention is not limited thereto and the monomers and the repetition numbers of the subscripts may be detected through various methods for recognizing the molecular structural formula.

In embodiments, the extracting the monomers and the repetition numbers of the subscripts that are detected from the polymer molecular structure formula image may use the method described with reference to FIGS. 7 to 11, but the present invention is not limited thereto.

The step of outputting the result (S1050) may generate a predetermined string format corresponding to the polymer molecular structure formula image based on the information about the plurality of atoms, the information about the bonding relationships between the plurality of atoms, and the cluster data.

In this case, the string format may include a mol file format, an sdf file format, etc., that are file formats that may represent information about compounds (e.g., element positions, bonding relationships, monomer information, etc.). The string format may include information about the simplified molecular input line entry system (SMILES).

The method for recognizing the polymer molecular structure formula according to embodiments of the present invention may be implemented by the system described with reference to FIGS. 1 and 2.

In addition, the disclosed embodiments may be implemented in the form of a recording medium in which computer-executable commands are stored. The commands may be stored in the form of program code, and when executed by the processor, program modules are generated to perform operations of the disclosed embodiments. The recording medium may be implemented as a computer-readable recording medium.

The computer-readable recording medium includes all types of recording media in which computer-decodable commands are stored. For example, such recording media may include read only memory (ROM), random access memory (RAM), a magnetic tape, a magnetic disk, a flash memory, an optical data storage device, and the like.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A system for recognizing a polymer molecular structural formula using artificial intelligence, the system comprising:
at least one processor; and
at least one memory storing a command or information that causes the at least one processor to perform an operation,
wherein the operation performed by the command or the information includes:
detecting a polymer molecular structure formula image to generate detection data including information about a bracket pair and a subscript associated with the bracket pair; and
inputting the detection data to each of a first model and a second model to output first cluster data from the first model and to output from the second model second cluster data including group information about the bracket pair and the subscript associated with the bracket pair, where the second cluster data includes information different from information provided by the first cluster data.

2. The system of claim 1, wherein the polymer includes two or more different monomers.

3. The system of claim 1, wherein the first cluster data includes at least one of class information and coordinate information of the bracket pair and the associated subscript.

4. The system of claim 1, wherein the second model includes a metric function.

5. The system of claim 1, wherein the detecting of the polymer molecular structure formula image to generate the detection data including the information about the bracket pair and the associated subscript includes inputting the polymer molecular structure formula image into a detector to output the detection data, and
the detector includes a detection transformer (DETR) or a deformable DETR.

6. The system of claim 1, wherein the detection data includes an embedding vector.

7. The system of claim 6, wherein at least one of the first model and the second model includes a matrix including the embedding vector.

8. The system of claim 2, wherein the group information about the bracket pair and the associated subscript includes information about which bracket pair and associated subscript among the detected bracket pairs and associated subscripts are included in a group corresponding to each monomer.

9. The system of claim 1, wherein the operation performed by the command or the information further includes converting the structural formula image into a predetermined string format including simplified molecular input line entry system (SMILES) based on cluster data and outputting the converted structural formula image.

10. The system of claim 1, wherein the operation performed by the command or the information further includes acquiring information about a plurality of atomic regions from the polymer molecular structure formula image and acquiring information about bonding relationships between a plurality of atoms based on the information about the plurality of atomic regions.

11. A method for recognizing a polymer molecular structural formula using artificial intelligence, performed by at least one processor, the method comprising:
detecting a polymer molecular structure formula image to generate detection data including information about a bracket pair and an associated subscript; and
inputting the detection data to each of a first model and a second model to output first cluster data from the first model and to output from the second model second cluster data including group information about the bracket pair and the associated subscript, where the second cluster data includes information different from information provided by the first cluster data.

12. The method of claim 11, wherein the polymer includes two or more different monomers.

13. The method of claim 12, wherein the group information about the bracket pair and the associated subscript includes information about which bracket pair and associated subscript among the detected bracket pairs and associated subscripts are included in a group corresponding to each monomer.

14. The method of claim 12, wherein the outputting of the second cluster data from the second model includes generating a matrix that projects the detection data into another feature space.

15. A program stored in a computer-readable recording medium to execute the method of claim 11 by being coupled to a computer.
